(19) United States
(12) Patent Application Publication
(10) Pub. No.: US 2017/0055903 A1
(43) Pub. Date: Mar. 2, 2017

(54) Electrode Holding Arrangement and Manufacturing Method Thereof

(71) Applicant: Stichting IMEC Nederland, Eindhoven (NL)

(72) Inventors: Peter Cramer, Eindhoven (NL); Bas Groenendaal, Eindhoven (NL)

(73) Assignee: Stichting IMEC Nederland, Eindhoven (NL)

(21) Appl. No.: 15/250,827

(22) Filed: Aug. 29, 2016

(30) Foreign Application Priority Data
Aug. 31, 2015 (EP) 15183098.1

(51) Int. Cl. A61B 5/00; A61N 1/04

(52) U.S. Cl. CPC A61B 5/6803; A61N 1/0484; A61B 2562/125

(57) ABSTRACT

The present disclosure describes methods and devices relating to electrode holding arrangements for bioelectric use. An example electrode holding arrangement includes a piece of flexible material configured to be fixed to a body part and at least one electrode spring element. The at least one electrode spring element is created by cutting out a certain geometric profile from the piece of flexible material. When the piece of flexible material is fixed to the body part having a body tissue surface, and an electrode is attached to the at least one electrode spring element and is positioned on the body tissue surface so that the body tissue surface generates a first force that makes the electrode spring element protrude outside a horizontal plane of the piece of flexible material, the at least one electrode spring element generates a second force that presses the electrode against the body tissue surface.

### CROSS-REFERENCE TO RELATED APPLICATIONS

The present application is a non-provisional patent application claiming priority to European Patent Application No. EP 15183098.1, filed Aug. 31, 2015, the contents of which are hereby incorporated by reference.

### FIELD OF THE DISCLOSURE

The present disclosure relates generally to the field of bioelectrical signal acquisition or stimulation devices and systems and more specifically to an electrode holding arrangement for such applications in which the electrode shall be pressed towards a certain tissue surface in order to make a stable and reliable electrical connection.

### BACKGROUND

Monitoring of biopotential signals, such as electrocardiogram (ECG), electroencephalogram (EEG) or electromyogram (EMG) signals and the use of electrostimulators for pain relief or injury recovery, is a highly relevant topic in personal healthcare. An important challenge in some application environments, for example when using dry electrodes, is to achieve a stable and reliable electrode-skin contact interface for improving the electrical signal quality for sensing or stimulation purposes.

Some example arrangements may include electrodes that are held and pressed, for example, towards the wrist, chest or scalp of a subject. One of such examples is patent application US 2013/0066184 A1, which describes a sensor headset with a contoured electrode that allows stable skin/head contact. U.S. Pat. No. 6,574,513 B1 also describes a mounting device for positioning and holding wrapped electrodes on a scalp. Another example of an arrangement for holding electrodes on the head of a person is described in U.S. Pat. No. 5,800,351.

### SUMMARY

The present description provides for an electrode holding arrangement with improved capabilities.

According to an exemplary embodiment, there is provided an electrode holding arrangement for bioelectric use, comprising: a piece of flexible material configured to be fixed to a certain body part; at least one electrode spring element; and wherein the at least one electrode spring element is created within the piece of flexible material by cutting out a certain geometric profile from said flexible material, and is configured to, when the piece of flexible material is fixed to a body part and an electrode is attached to the at least one electrode spring element and is positioned on a body tissue surface so that it generates a force that makes the electrode spring element protrude outside the horizontal plane of the plastic piece of flexible material, generate a force that presses said electrode against said body tissue surface.

According to an exemplary embodiment, the electrode holding arrangement comprises one or more electrode spring elements that can be created by simply cutting away a certain profile of material from a piece of flexible material, which may be for example a single sheet of plastic that is also cut in a form that adapts to the required electrode applications (e.g. in the form of a headset, head, chest or wrist band or a patch). Therefore, the electrode holding arrangement may be manufactured from one sheet of flexible material, such as for example a flat sheet of plastic, using conventional cutting techniques, such as laser cutting, water jets, scissors or 3-D printing, which makes it very easy and cheap to manufacture. Furthermore, such an electrode holding arrangement may be compact and comprise less moving parts which, among other aspects, may reduce complexity of manufacturing, assembly and mounting time. Since it is compact and can be made with lightweight material, the negative effects of motion artifacts may also be reduced. In some examples, manufacturing of the electrode spring elements requires only one operation and is therefore cost efficient for any quantity, which allows for individual customization. For example, the electrode spring elements can be easily manufactured to obtain a certain desired constant and/or well controlled force or pressure applied on the electrodes: such force can be very efficiently controlled, since the electrode will tend to have a constant displacement once the arrangement is adjusted and the amount of force can be easily adapted and optimized by changing the flexible material properties and/or the geometry (the cutting profile pattern) of the electrode spring elements. In some examples, the electrode spring element may be designed such that the direction of compression of the electrode is perpendicular to the body tissue surface, which may improve the electrical signal quality. This is particularly useful with dry electrodes and for applications that require that those electrodes are pressed to the body tissue for long periods of time without interfering with normal daily activities. According to an exemplary embodiment, the electrode holding arrangement is suited for the manufacture of EEG headsets, since the flexible material sheet from which the electrode spring element is made can constitute the base part of the EEG cap to which the electronic components may be later applied. Furthermore, the electrode position, deflection and pressure can be easily adapted to different head sizes and shapes.

According to an exemplary embodiment, the at least one electrode spring element comprises at least: a first and a second connection arrangement connected to a middle segment piece; and wherein the at least first and second connection arrangement connects a different portion of the middle segment piece to a different anchor point of the piece of flexible material.

According to an exemplary embodiment, the at least first and second connection arrangement comprise at least one extendable element. According to an exemplary embodiment, the extendable element comprises a segment with a curved or meander shape. According to another exemplary embodiment, the extendable element comprises connected U-shaped segments. The connected U-shaped segments may have different lengths.

According to an exemplary embodiment, the middle segment piece may have an opening for fixing an electrode to the electrode spring element. According to another exemplary embodiment, the electrode spring element further comprises an electrode holder.

According to an exemplary embodiment, the piece of flexible material is made of a plastic, cardboard or a plastic-like material, e.g. a material with similar performance as a plastic.

According to an exemplary embodiment, the piece of flexible material may be cut in a form that fits a certain body part.

According to an exemplary embodiment, the electrode holding arrangement further comprises fixing and/or fitting means to secure the piece of flexible material to a certain body part.

According to an exemplary embodiment, the electrode holding arrangement according to embodiments may be used as a headset, headband, wristband or body arrangement for bioelectric sensing or stimulation.

Some embodiments may relate to a wearable device or garment comprising an electrode holding arrangement according to embodiments described herein.

Other embodiments may relate to a method for manufacturing an electrode holding arrangement comprising: cutting a piece of flexible material into a form that fits a certain body part; cutting out a certain geometric profile from said flexible material in order to create at least one electrode spring element within said piece of flexible material; and wherein the at least one electrode spring element is configured to, when the piece of flexible material is fixed to a body part and an electrode is attached to the at least one electrode spring element and is positioned on a body tissue surface so that it generates a force that makes the electrode spring element protrude outside the horizontal plane of the plastic piece of flexible material, generate a force that presses said electrode against said body tissue surface.

Certain aspects of various embodiments have been described above. It is to be understood that not necessarily all such aspects may be achieved in accordance with any particular embodiment in the present disclosure. Those skilled in the art will recognize that the embodiments described herein may be embodied or carried out in a manner that achieves or optimizes one aspect or group of aspects without necessarily achieving other objects or aspects.

### BRIEF DESCRIPTION OF THE DRAWINGS

The above and other aspects of the electrode holding arrangement and manufacturing method thereof will be shown and explained with reference to the non-restrictive example embodiments described hereinafter.

FIGS. 1A and 1B show a top and side view of a general schematic representation of an electrode holding arrangement comprising a piece of flexible material and an electrode spring element according to a first exemplary embodiment.

FIGS. 2A and 2B show a side view of the electrode holding arrangement of FIG. 1 comprising an electrode and with the electrode spring element in a neutral state and in operation according to exemplary embodiments.

FIG. 3 shows a perspective view of the electrode holding arrangement of FIG. 2B when in operation according to an exemplary embodiment.

FIG. 4 shows a top view of a general schematic representation of another electrode holding arrangement according to a second exemplary embodiment.

FIG. 5 shows a top view of an exemplary implementation of an electrode spring element according to a third embodiment.

FIGS. 6A and 6B show a side view of the electrode holding arrangement of FIG. 4 comprising an electrode and with the electrode spring element in a neutral state and in operation according to exemplary embodiments.

FIG. 7 shows a top view of an exemplary implementation of an electrode spring element according to a fourth embodiment.

FIG. 8 shows a top view of another electrode spring element according to a fifth exemplary embodiment.

FIG. 9 shows a top view of another electrode spring element according to a sixth exemplary embodiment.

FIG. 10 shows a top view of another electrode spring element according to a seventh exemplary embodiment.

FIGS. 11A and 11B show a top view of further exemplary embodiments of electrode spring elements.

FIG. 12 shows an exemplary electrode holding arrangement in the form of a headset device.

FIG. 13 shows an exemplary electrode holding arrangement in the form of a headband.

FIG. 14 shows an exemplary electrode holding arrangement in the form of a wristband.

### DETAILED DESCRIPTION OF ILLUSTRATIVE EMBODIMENTS

In the following, in the description of exemplary embodiments, various features may be grouped together in a single embodiment, figure, or description thereof for the purpose of streamlining the disclosure and aiding in the understanding of one or more of the various inventive aspects. This is however not to be interpreted as any embodiment requiring more features than the ones expressly recited in the independent claims. Furthermore, combinations of features of different embodiments are meant to be within the scope of the present description, as would be clearly understood and derived by those skilled in the art. Additionally, in some examples, well-known methods, structures and techniques have not been shown in detail in order not to obscure the conciseness of the description.

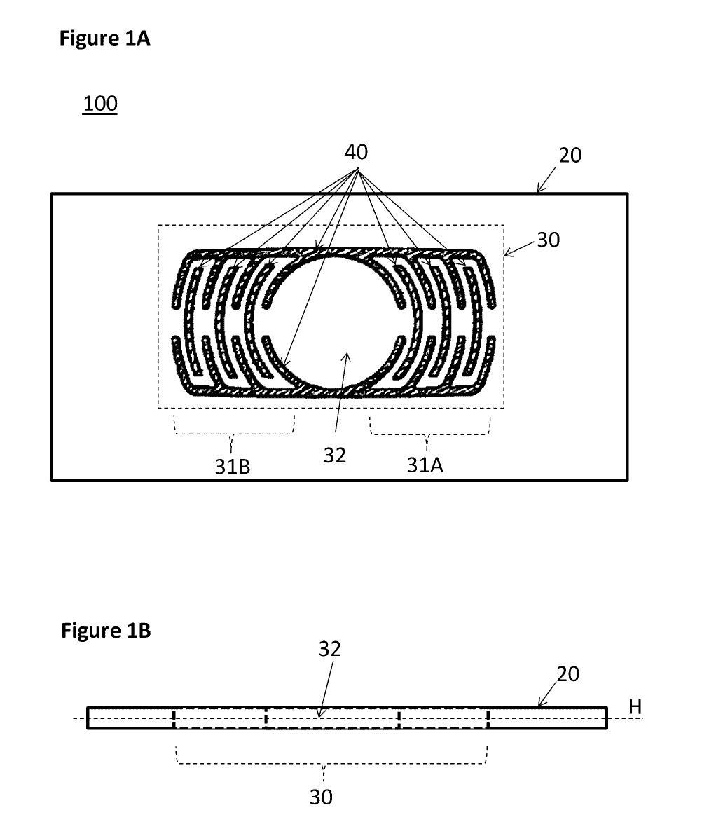

FIG. 1A shows a top view of a general schematic representation of an electrode holding arrangement 100 according to a first exemplary embodiment. The electrode holding arrangement 100 may be for example a wrist band, a headband, a headset or any other arrangement or structure that shall hold one or more electrodes pressed towards a certain tissue surface in order to make a stable and reliable electrical connection. The electrode holding arrangement 100 may comprise a piece of flexible material 20, such as a plastic material, in which at least one electrode spring element 30 is generated. The electrode spring element 30 may be created, for example, by cutting a profile 40 (indicated in the figure by a diagonal pattern profile) of the piece of flexible material 20. Such cut profile may for example define the contour of the electrode spring element 30. According to an exemplary embodiment, the contour may be cut out of a thin plate of the piece of flexible material 20 by means of conventional cutting, laser cutting or cutting with a high-pressure jet stream. According to exemplary embodiments, the electrode holding arrangement 100 may comprise just one piece of flexible material 20, but may also comprise a number of pieces of flexible material 20 assembled together. The piece or pieces of flexible material 20 may be for example straps or plates of variable forms and with a thickness between 0.2 to 5 mm. A side view of such strap or plate can be seen in FIG. 1B in which the electrode spring element 30 is in neutral state, that is, not in operation. According to exemplary embodiments, the piece or pieces of flexible material 20 may be made of a plastic, but could also be made of cardboard or other materials with flexible characteristics and performance similar to plastic.

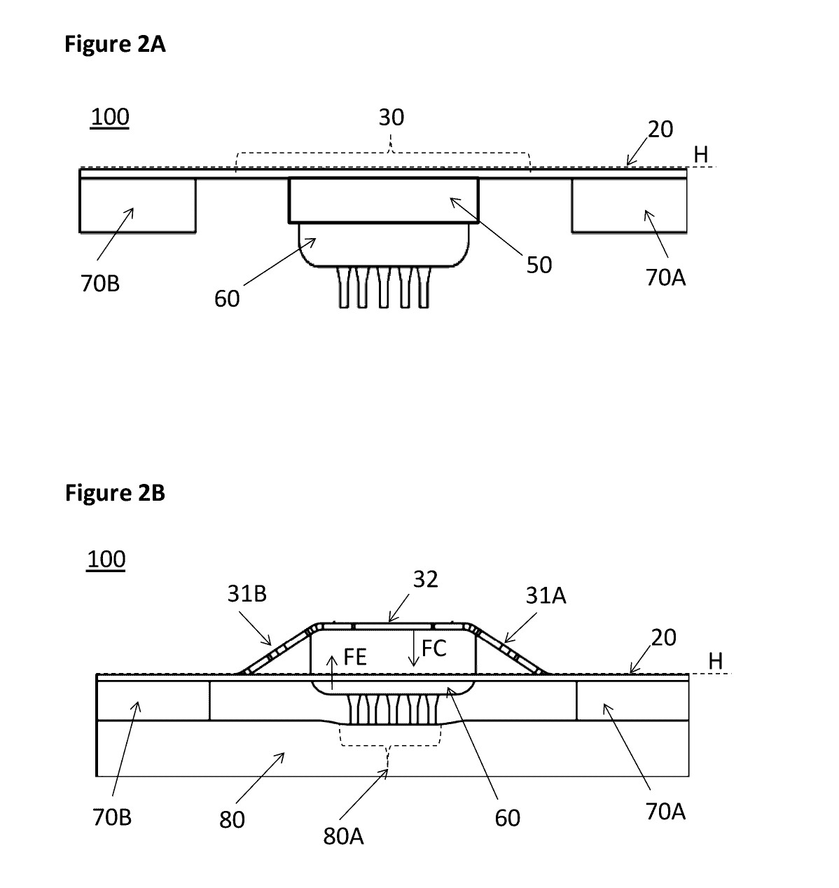

The electrode spring element 30 is therefore part of, and made from and within, the piece of flexible material 20, e.g. by cutting out pieces of that flexible material. According to an exemplary embodiment, the electrode spring element 30 comprises at least a first connection arrangement 31A, a middle segment piece 32 and a second connection arrangement 31B and its geometry is designed to provide that element with a spring property or action such that an electrode placed under the middle segment piece 32 is pressed towards the body tissue surface in that location. According to an exemplary embodiment, the first and second connection arrangements 31A and 31B of the electrode spring element 30 connect a different side of the middle segment piece 32 to a different anchor point of the piece of flexible material 20 and such that, in operation (as shown in FIG. 2B), that is, when an electrode 60 is positioned under the middle segment piece 32 and over a tissue surface 80 and generates a force that makes that middle segment piece 32 protrude outside the horizontal plane H of the sheet of flexible material 20, the first and second connection arrangements 31A, 31B generate a compression force that pulls the middle segment piece 32 and hence forces the electrode towards that tissue surface. According to an exemplary embodiment the first and second connection arrangements 31A, 31B of the electrode spring element 30 have the same geometry and connect an opposite side of the middle segment piece 32. According to an exemplary embodiment, the first and second connection arrangements 31A, 31B may be designed such that the compression force is perpendicular to the tissue surface. This maintains the electrode perpendicular to the tissue surface and helps improve the electrode-skin contact, which assures better electrical signal quality transfer. According to an exemplary embodiment the first and second connection arrangements 31A, 31B comprise a plurality of extendable elements in the form of connected U-shaped segments. According to exemplary embodiments the middle segment piece 32 has a circular or substantially circular shape, but other shapes are possible, like for example elliptical or orthogonal shapes.

It should be understood that the force by which the electrode is pressed to the tissue may depend on the flexible material characteristics and the geometry of the electrode spring element 30, and more specifically to the implementation of the design of the first and second connection arrangements 31A and 31B. The number of extendable elements of the first and second connection arrangements that connect the middle segment piece 32 to the piece of flexible material 20 and their form will also play an important role in achieving the desired pressure performance characteristics.

FIG. 2A shows a side view of the electrode holding arrangement 100 of FIG. 1 comprising a piece of flexible material 20 and an electrode 60 attached to an electrode spring element 30 formed within the piece of flexible material. According to exemplary embodiments, the electrode 60 may be attached directly to the middle segment piece 32 of the electrode spring element 30 or attached to an electrode holder or retainer 50 which is fixed to that middle segment piece 32. The electrode spring element 30 of the figure is in neutral position as it does not protrude outside of the horizontal plane H of the sheet of flexible material 20. According to an exemplary embodiment, the electrode holding arrangement 100 may further comprise a plurality of foam layers 70A, 70B attached to the inner side of the plastic sheet piece of flexible material 20.

FIG. 2B shows a side view of the electrode holding arrangement 100 of FIGS. 1A and 2A and in which the electrode spring element 30 is in operation, that is, when the electrode 60 is positioned under the middle segment piece 32 and on the surface of a body tissue 80, it generates a force FE that makes the middle segment piece 32 protrude outside the horizontal plane H of the plastic piece of flexible material 20, and the first and second connection arrangements 31A, 31B generate a compression force FC that pulls the middle segment piece 32 and hence the electrode 60 towards that body tissue surface location 80A. According to an exemplary embodiment, the electrode holding arrangement 100 also comprises a plurality of foam layers 70A, 70B attached between the inner side of the plastic sheet piece of flexible material 20 and the body tissue 80 in order to help maintain a certain distance between the electrode and the tissue surface, and to provide additional comfort to the electrode holding arrangement wearer subject.

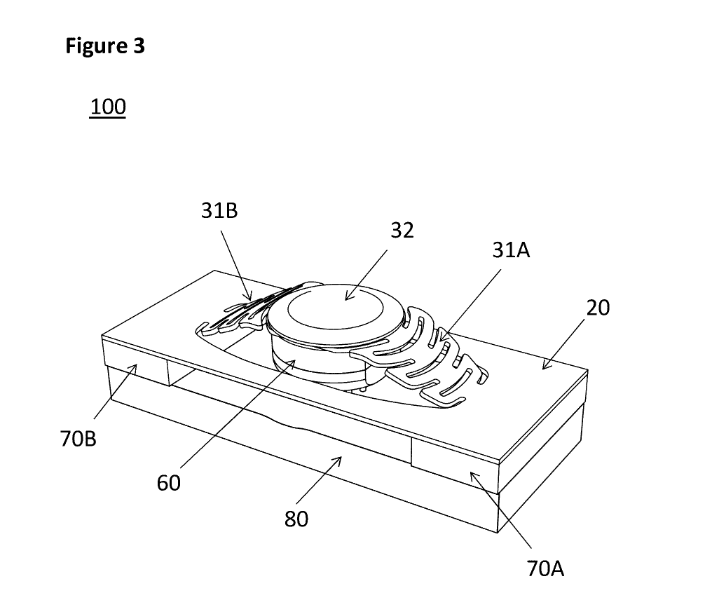

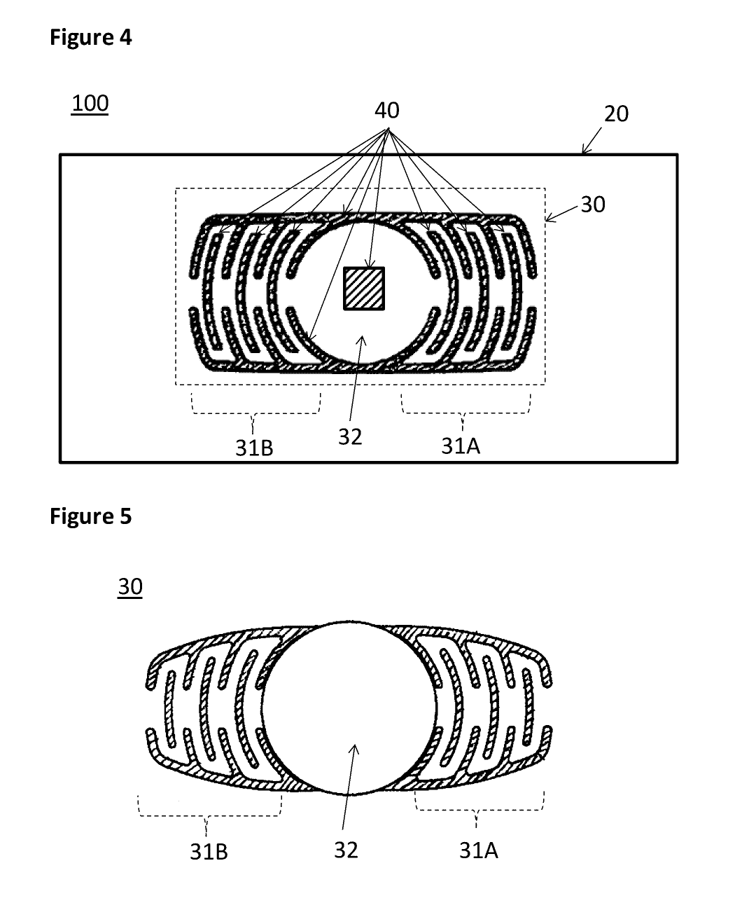

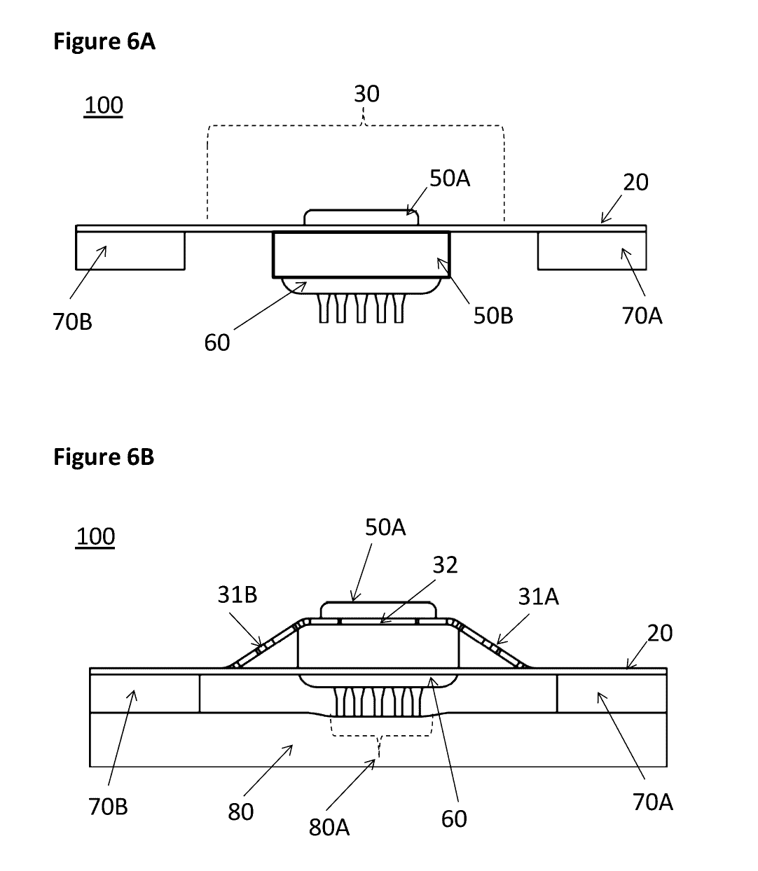

A perspective view of an electrode holding arrangement 100 according to an exemplary embodiment, when the electrode spring element 30 is in operation, is shown in FIG. 3. In particular, the exemplary embodiment of the electrode spring element is described in FIG. 5. FIG. 4 shows a top view of a general schematic representation of an electrode holding arrangement 100 according to a second exemplary embodiment. The electrode holding arrangement 100 may be similar or identical to the arrangement shown in FIG. 1A, with the difference that the cut profile 40 and hence the electrode spring element 30 provides for an opening in the middle segment piece 32. Such an opening may have variable shapes and may be intended for providing further connection functionality between the middle segment piece 32 and the electrode holder or retainer 50. For example, instead of attaching the electrode holder or retainer 50 to one side (the inner side towards the tissue surface) of the middle segment piece 32 as shown in FIG. 2A, such an electrode holder may comprise two holding elements 50A, 50B connected through the opening of the middle segment piece 32 as shown in FIG. 6A, which may provide improved connectivity between the electrode spring element 30 and the electrode 60. FIG. 6B shows the electrode holding arrangement 100 of FIGS. 4 and 6A in operation, similar to what is shown in FIGS. 2B and 3.

FIG. 5 shows a top view of another exemplary implementation of an electrode spring element 30 according to a third exemplary embodiment. In this embodiment the first and second connection arrangements 31A, 31B comprise a plurality of extendable elements in the form of connected U-shaped segments and in which the U-shaped segments do not have the same length, that is, the extendable elements vary in size. According to another exemplary embodiment the middle segment piece 32 of the electrode spring element 30 may also comprise an opening as shown in FIG. 4.

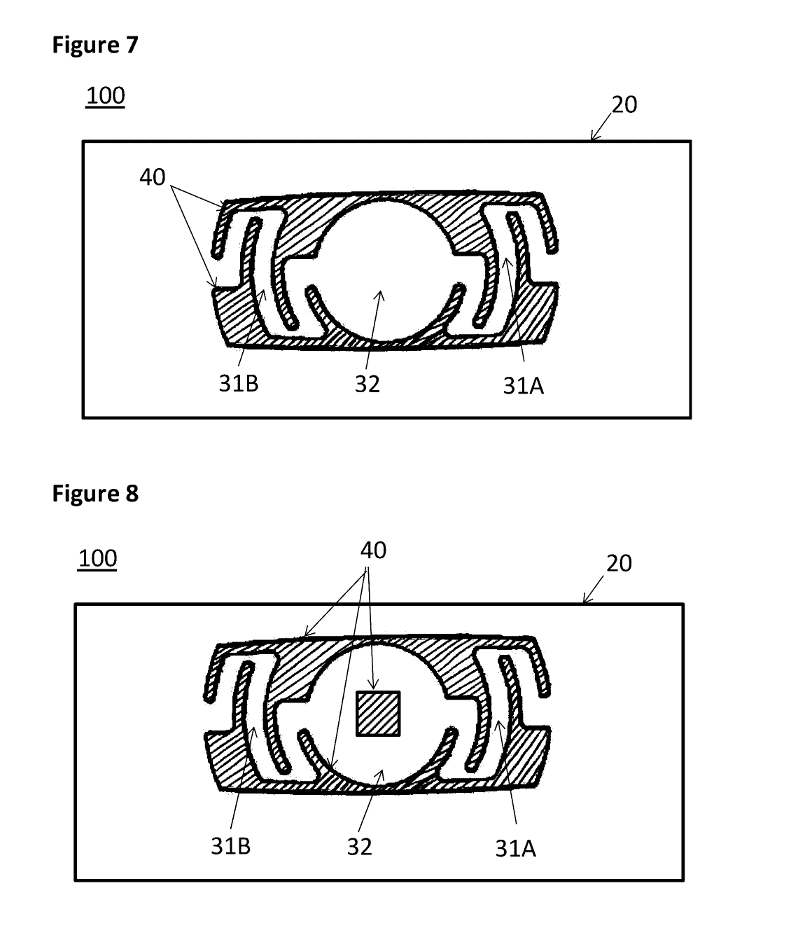

FIG. 7 shows a top view of a general schematic representation of an electrode holding arrangement 100 according to a fourth exemplary embodiment, comprising a piece of flexible material 20, such as a plastic material, in which at least one electrode spring element 30 is generated. The electrode spring element 30 may be created, for example, by cutting a profile 40 (indicated in FIG. 7 by a diagonal pattern profile) of the piece of flexible material 20. According to an exemplary embodiment, the electrode spring element 30 comprises at least a first connection arrangement 31A, a middle segment piece 32 and a second connection arrangement 31B and its geometry is designed to provide that element with a spring property or action such that an electrode placed under the middle segment piece 32 is pressed towards the body tissue surface in that location. According to an exemplary embodiment, the first and second connection arrangements 31A, 31B of the electrode spring element 30 connect a different side of the middle segment piece 32 to the piece of flexible material 20. In this exemplary embodiment, the first and second connection arrangements 31A, 31B comprise a curved segment connection or in the form of a meander. According to exemplary embodiments the middle segment piece 32 has a circular or substantially circular shape, but other shapes are possible, like for example elliptical or orthogonal shapes. FIG. 8 shows a top view of a general schematic representation of another electrode holding arrangement 100 according to a fifth exemplary embodiment, which may be similar or identical to the electrode holding arrangement 100 of FIG. 7 but with the middle segment piece 32 of the electrode spring element 30 also having an opening for improved fixation to an electrode or electrode holder.

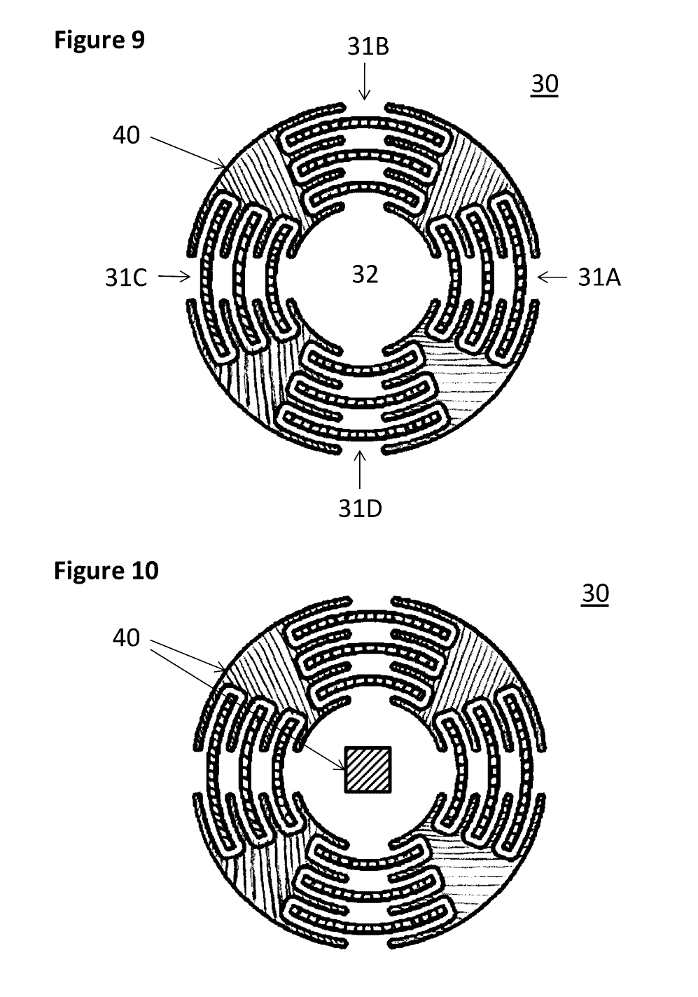

FIG. 9 shows a top view of another exemplary implementation of an electrode spring element 30 according to a sixth exemplary embodiment wherein the cut profile 40 provides for a circularly shaped electrode spring element geometry. In this embodiment the electrode spring element 30 comprises four connection arrangements 31A, 31B, 31C, 31D that connect the middle segment piece 32 to different anchor points of the piece of flexible material 20 (not shown). The four connection arrangements comprise a plurality of extendable elements in the form of connected U-shaped segments and in which the U-shaped segments do not have the same length. FIG. 10 shows basically the same electrode spring element 30 as the one shown in FIG. 9 but now the middle segment piece 32 of the electrode spring element 30 has also an opening for improved fixation to an electrode or electrode holder.

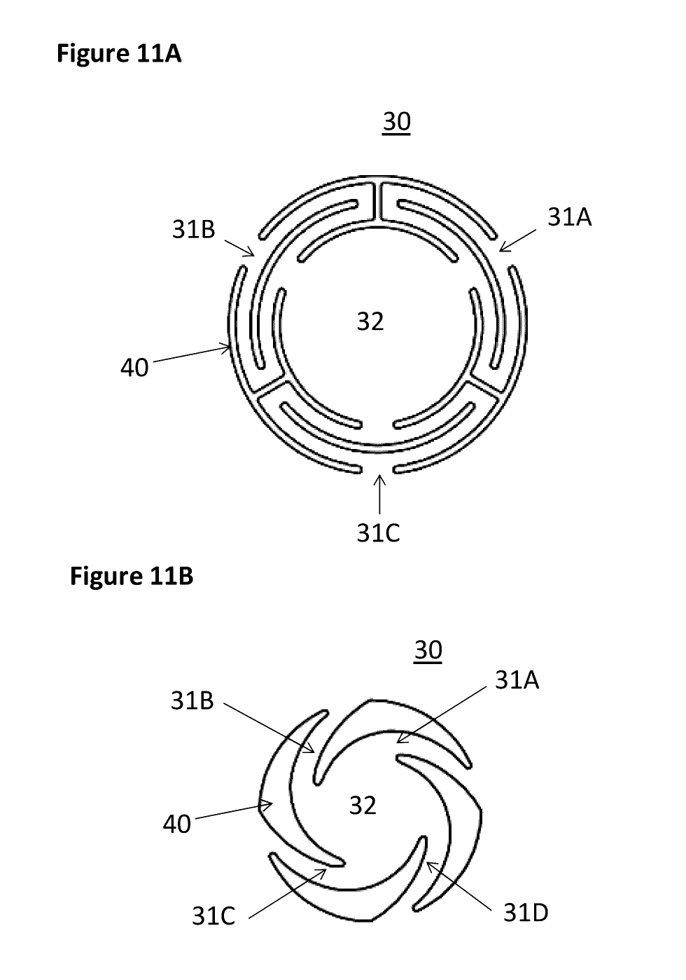

FIG. 11A shows a top view of another exemplary implementation of an electrode spring element according to an eighth exemplary embodiment. In this embodiment the electrode spring element 30 and cut profile 40 provide for a circular geometry and comprises three connection arrangements 31A, 31B, 31C that connect the middle segment piece 32 to the piece of flexible material 20. The three connection arrangements comprise one extendable element in the form of connected U-shaped segments. The connection arrangements in this exemplary embodiment circumvent the middle segment piece 32. FIG. 11B shows a top view of another exemplary implementation of an electrode spring element according to a ninth exemplary embodiment. In this embodiment the electrode spring element 30 and cut profile provide for a substantially circular geometry and comprises four connection arrangements 31A, 31B, 31C, 31D that connect the middle segment piece 32 to the piece of flexible material 20. The four connection arrangements comprise one extendable element in the form of curved segments. According to exemplary embodiments, the middle segment piece 32 of the electrode spring elements of FIGS. 11A and 11B may also comprise an opening as shown for example in FIGS. 4, 8 and 10 for improved fixation to an electrode or electrode holder.

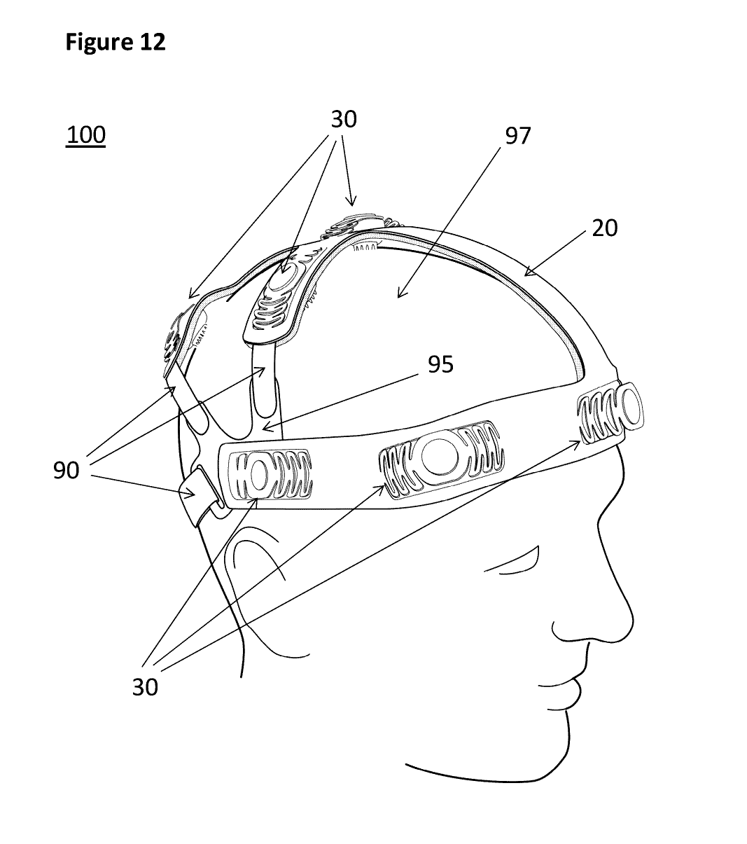

FIG. 12 shows an exemplary electrode holding arrangement 100 in the form of a headset device, showing a plurality of electrode spring elements 30 implemented in a single piece of flexible material 20. According to an exemplary embodiment, the single piece of flexible material is also cut in a form that fits the head 97 of a subject and can be fixed and secured to the subject's head by means of fixing straps 90 and one or more fittings 95. According to an exemplary embodiment, the cut out of the plastic sheet form and the electrode spring elements may be done at the same time and in a single step. Electrodes may be fixed to the electrode spring elements for sensing purposes. The headset may comprise a foam layer attached between the inner side of the plastic sheet piece of flexible material 20 and the scalp in order to help maintain a certain distance between the electrodes and the head surface, and to provide additional wearing comfort to the subject.

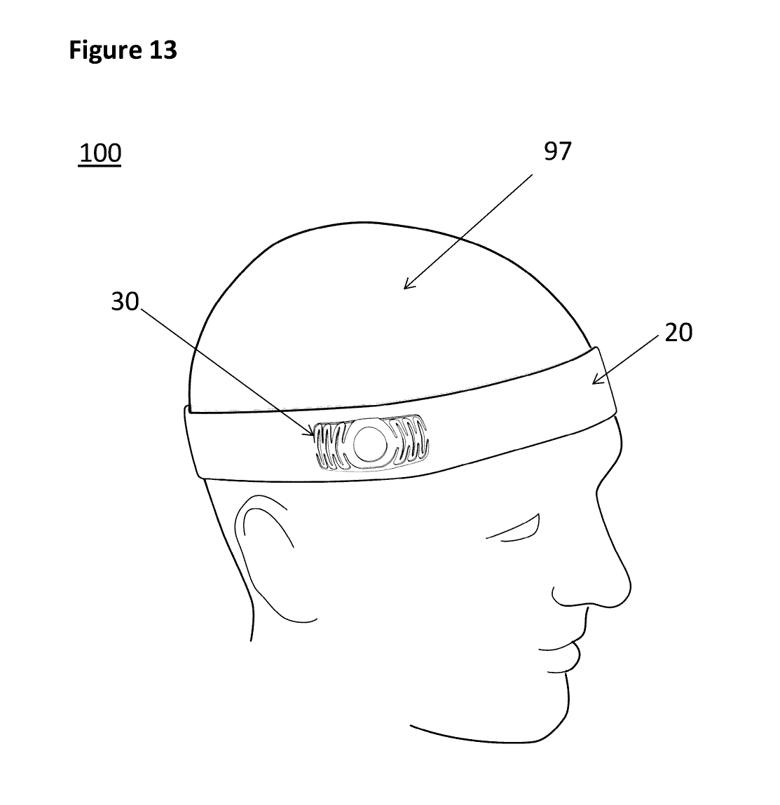

FIG. 13 shows another exemplary electrode holding arrangement 100 in the form of a headband, made of a single piece of flexible material 20, e.g. a plastic sheet that has been cut in the form of a headband, and in which at least one electrode spring element 30 has been generated. According to an exemplary embodiment, the cut out of the plastic sheet form and the electrode spring element may be done at the same time and in a single step. An electrode may be fixed to the electrode spring element for sensing purposes. The headband may also comprise a foam layer attached between the inner side of the plastic sheet piece of flexible material and the scalp in order to help maintain a certain distance between the electrode and the skin surface, and to provide additional wearing comfort to the subject. According to an exemplary embodiment, each side of the single piece of flexible material may be connected together by known fixing or fitting means and then fitted and secured to the head 97 of the subject.

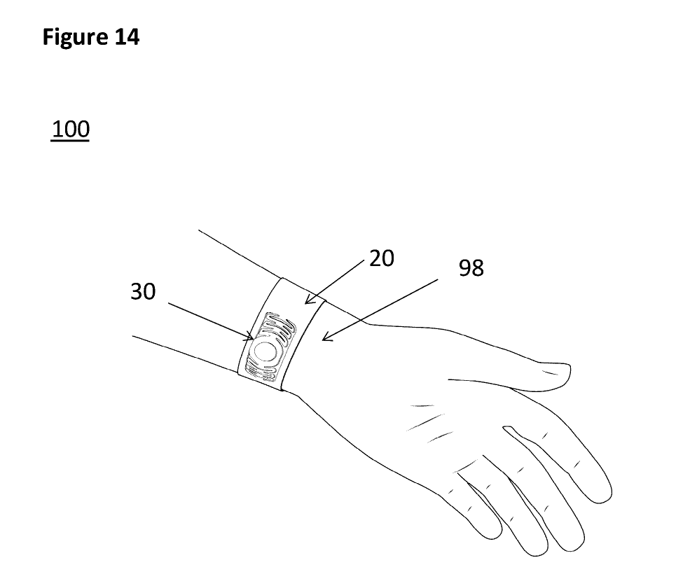

FIG. 14 shows another exemplary electrode holding arrangement 100 in the form of a wristband, made of a single piece of flexible material 20, e.g. a plastic sheet that has been cut in the form of a wristband, and in which at least one electrode spring element 30 has been generated. According to an exemplary embodiment, the cut out of the plastic sheet form and the electrode spring element may be done at the same time and in a single step. An electrode may be fixed to the electrode spring element for sensing purposes. The wristband may also comprise a foam layer or other material attached between the inner side of the piece of flexible material and the wrist skin in order to help maintain a certain distance between the electrode and the skin surface, and to provide additional wearing comfort to the subject. According to an exemplary embodiment, each side of the single piece of flexible material may be connected together by known fixing or fitting means and then fitted and secured to the wrist 98 of the subject.

1. An electrode holding arrangement for bioelectric use, comprising:
a piece of flexible material configured to be fixed to a body part; and
at least one electrode spring element;
wherein the at least one electrode spring element is created within the piece of flexible material by cutting out a certain geometric profile from the piece of flexible material, and wherein the at least one electrode spring element is configured to:
when the piece of flexible material is fixed to the body part having a body tissue surface, and an electrode is attached to the at least one electrode spring element and is positioned on the body tissue surface so that the body tissue surface generates a first force that makes the electrode spring element protrude outside a horizontal plane of the piece of flexible material, generate a second force that presses the electrode against the body tissue surface.

2. An electrode holding arrangement according to claim 1, wherein the at least one electrode spring element comprises:
a first connection arrangement and a second connection arrangement connected to a middle segment piece, wherein the first and second connection arrangements connect different portions of the middle segment piece to respective anchor points of the piece of flexible material.

3. An electrode holding arrangement according to claim 2, wherein the first and second connection arrangements each comprise at least one extendable element.

4. An electrode holding arrangement according to claim 3, wherein the extendable element comprises a segment with a curved or meander shape.

5. An electrode holding arrangement according to claim 3, wherein the extendable element comprises connected U-shaped segments.

6. An electrode holding arrangement according to claim 5, wherein the connected U-shaped segments have different lengths.

7. An electrode holding arrangement according to claim 2, wherein the middle segment piece has an opening for fixing an electrode to the electrode spring element.

8. An electrode holding arrangement according to claim 1, wherein the electrode spring element further comprises an electrode holder.

9. An electrode holding arrangement according to claim 1, wherein the piece of flexible material is plastic.

10. An electrode holding arrangement according to claim 1, wherein the piece of flexible material is cut in a form that fits a certain body part.

11. An electrode holding arrangement according to claim 1, further comprising a fixing material to secure the piece of flexible material to a certain body part.

12. An electrode holding arrangement according to claim 1, wherein the arrangement is configured to be used in a one of a headset, a headband, or a wristband for bioelectric sensing or stimulation.

13. An electrode holding arrangement according to claim 1, wherein the electrode spring element comprises four connection arrangements connected to a middle segment piece, wherein each of the four connection arrangements are configured to connect a different respective portion of the middle segment piece to a respective anchor point of the piece of flexible material.

14. A wearable device, comprising:
a piece of flexible material configured to be fixed to a body part; and
at least one electrode spring element;
wherein the at least one electrode spring element is created within the piece of flexible material by cutting out a certain geometric profile from the piece of flexible material, and wherein the at least one electrode spring element is configured to:
when the piece of flexible material is fixed to the body part having a body tissue surface, and an electrode is attached to the at least one electrode spring element and is positioned on the body tissue surface so that the body tissue surface generates a first force that makes the electrode spring element protrude outside a horizontal plane of the piece of flexible material, generate a second force that presses the electrode against the body tissue surface.

15. A method for manufacturing an electrode holding arrangement, comprising:
cutting a piece of flexible material into a form that fits a certain body part; and
cutting out a certain geometric profile from the piece of flexible material in order to create at least one electrode spring element within the piece of flexible material;
wherein the at least one electrode spring element is configured to, when the piece of flexible material is fixed to the certain body part and an electrode is attached to the at least one electrode spring element and is positioned on a body tissue surface of the certain body part such that the body tissue surface generates a force that makes the electrode spring element protrude outside a horizontal plane of the piece of flexible material, generate a force that presses the electrode against the body tissue surface.

16. The method of claim 15, wherein the certain geometric profile comprises connected U-shaped segments.

17. The method of claim 16, wherein the U-shaped segments have different lengths.

18. The method of claim 15, wherein the certain geometric profile comprises curved or meander shaped segments.

19. The method of claim 15, further comprising cutting the piece of flexible material to include an opening through which an electrode is fixed to the electrode spring element.

20. The method of claim 19, wherein the opening is in a center portion of the electrode spring element.
